(19) United States
(12) Patent Application Publication
(10) Pub. No.: US 2024/0298648 A1
(43) Pub. Date: Sep. 12, 2024

(54) MICROBIAL AGENT WITH FUNCTIONS OF PREVENTING AND CONTROLLING AFLATOXIN AND AFLATOXIGENIC STRAIN THEREOF AND PROMOTING YIELD INCREASE OF CROPS AND USE THEREOF

(71) Applicant: OIL CROPS RESEARCH INSTITUTE, CHINESE ACADEMY OF AGRICULTURAL SCIENCES, Hubei (CN)

(72) Inventors: Qi ZHANG, Hubei (CN); Peiwu LI, Hubei (CN); Xiaofeng YUE, Hubei (CN); Xiaoqian TANG, Hubei (CN); Yang ZHOU, Hubei (CN); Yizhen BAI, Hubei (CN)

(73) Assignee: OIL CROPS RESEARCH INSTITUTE, CHINESE ACADEMY OF AGRICULTURAL SCIENCES, Hubei (CN)

(21) Appl. No.: 18/664,303

(22) Filed: May 15, 2024

Related U.S. Application Data
(63) Continuation of application No. PCT/CN2022/131943, filed on Nov. 15, 2022.

(30) Foreign Application Priority Data
Nov. 15, 2021 (CN) 202111344662.7

(51) Int. Cl. A01N 63/22; A01N 25/08; A01N 25/12; C12N 1/20; C12R 1/07; C12R 1/13

(52) U.S. Cl. CPC A01N 63/22; A01N 25/08; A01N 25/12; C12N 1/20; C12R 2001/07; C12R 2001/13

(57) ABSTRACT

The present disclosure relates to a microbial agent with functions of preventing and controlling aflatoxin and aflatoxigenic strain thereof and promoting yield increase of crops. The microbial agent is compounded from 5 microbes comprising Bacillus amyloliquefaciens, Brevibacillus laterosporu, Bacillus mucilaginosus Krassilnikov, Enterobacter ludwigii and Myroides odoratimimus by separate fermentative cultivation, concentration and mixing. The microbial agent is applied to a field planting stage of crops such as peanuts, which can effectively reduce the abundance and infection probability of toxin-producing strain such as Aspergillus flavus in soil from the source, reduce the risk of aflatoxin pollution of peanuts after production, improve the quality and safety level of peanuts, and at the same time, promote crop growth, enhance the resistance, and improve the full pod rate and yield, and has significant economic, social and ecological benefits.

### CROSS-REFERENCE TO RELATED APPLICATION

The present application is a continuation of PCT application No. PCT/CN2022/131943, filed on Nov. 15, 2022, which claims the priority benefits of China Application No. 202111344662.7, filed on Nov. 15, 2021. The entirety of each of the above mentioned patent applications is incorporated by reference herein and made a part of this specification.

### BACKGROUND

### Technical Field

The present disclosure belongs to the field of microbial technology, and in particular relates to a microbial agent with functions of preventing and controlling aflatoxin and aflatoxigenic strain thereof and promoting yield increase of crops and use thereof.

### Description of Related Art

Aflatoxins (AFTs) are the most toxic mycotoxins found to pollute agricultural products so far, have acute and chronic toxicity and can cause cancer, teratogenicity, and mutagenesis.

Aflatoxins are mainly produced under suitable conditions after host plants are infected with Aspergillus fungi such as Aspergillus flavus and Aspergillus parasiticus. Common species include aflatoxin B₁ (AFB₁), aflatoxin B₂ (AFB₂), aflatoxin G₁ (AFG₁), and aflatoxin G₂ (AFG₂). Aflatoxin B₁ is the most toxic and the most carcinogenic. Its toxicity is 10 times that of potassium cyanide, and 68 times that of white arsenic, and its carcinogenicity is 10,000 times that of hexachlorocyclohexane, and Aflatoxin B₁ is listed as a class I carcinogen by the International Agency for Research on Cancer (IARC) of the World Health Organization (WHO), and there have been many deaths of human and animal poisoning caused by excessive aflatoxin at home and abroad. Aflatoxins are also pollutants that pollute the most types of food and agricultural products, and according to the Web of Science search data statistics over the last 5 years: aflatoxins pollute more than 110 types of foods and raw materials, ranking first among pollutants, with agricultural products such as peanuts being the most susceptible to pollution. Aflatoxin pollution can occur in the whole process of peanut field production, harvesting, storage and transportation. Establishing a field prevention and control technology for peanut aflatoxin pollution can reduce Aspergillus flavus infection and aflatoxin pollution from the source, which is of great significance for ensuring the quality and safety of agricultural products such as peanuts in China, and promoting the green and high-quality development of the industry.

Biocontrol using antagonistic microorganisms has the advantages of not destroying the quality of agricultural products, safety and high efficiency, and environmental friendliness, and is an effective way for green control of Aspergillus flavus and aflatoxin. However, the use and promotion of biological control in farmlands are restricted due to the ecological adaptability of strains, as well as inappropriate preparations or incapability of effectively playing the role of biocontrol. A large number of studies have been carried out in the screening of biocontrol strains of Aspergillus flavus in China. Strains such as bacteria, yeasts and molds with the effects of resisting bacteria or aflatoxin degradation have been identified in indoor or field experiments, however, there is currently no product that can be widely promoted and applied in production with significant bacterial and toxin reducing effects. In the prevention and control of aflatoxin in countries such as the United States and Argentina, non-toxin-producing Aspergillus flavus spores are inoculated on barley seeds and rice grains to be applied to a field, competing to inhibit the growth and reproduction of toxin-producing Aspergillus flavus to achieve the purpose of biocontrol. However, this method is high in cost, cumbersome to operate, and difficult to promote, and there is an urgent need for a biocontrol preparation product which has high efficiency, and is easy to store and promote, not only can effectively prevent and control aflatoxin pollution, but also has a promoting effect on increasing the crop yield.

An object of the present disclosure is to develop and apply a microbial agent in view of the deficiencies in the prior art, by antagonizing the growth of Aspergillus flavus, improving the structures of microbial populations in peanut rhizospheres, and optimizing the microecological environment, not only can the abundance and infection probability of aflatoxigenic strain such as Aspergillus flavus in soil be reduced from the source, and the aflatoxin pollution of peanuts be effectively controlled, but also the number of root nodules can be increased, the resistance of crops can be enhanced, and the full pod rate and yield can be improved. The microbial agent of the present disclosure is of great significance in improving the quality and safety level of agricultural products such as peanuts, realizing increasing crop yield and efficiency, reducing fertilizer application in farmlands, improving the ecological environment, and the like.

### SUMMARY

In view of the deficiencies in the prior art, the technical problem to be solved by the present disclosure is to provide a microbial agent with functions of preventing and controlling aflatoxin and aflatoxigenic strain thereof and promoting yield increase of crops and use thereof. The microbial agent is applied to a field planting stage of crops such as peanuts, which can effectively reduce the abundance and infection probability of toxin-producing strain such as Aspergillus flavus in soil from the source, reduce the risk of aflatoxin pollution of peanuts after production, improve the quality and safety level of peanuts, and at the same time, promote crop growth, enhance the resistance, and improve the full pod rate and yield, and has significant economic, social and ecological benefits.

To solve the above technical problems, the technical solutions adopted by the present disclosure are as follows:

provided is a microbial agent with functions of preventing and controlling aflatoxin and aflatoxigenic strain thereof and promoting yield increase of crops, compounded from 5 microbes comprising Bacillus amyloliquefaciens, Brevibacillus laterosporu, Bacillus mucilaginosus Krassilnikov, Enterobacter ludwigii and Myroides odoratimimus by separate fermentative cultivation, concentration and mixing.

Further, the Bacillus amyloliquefaciens is a Bacillus amyloliquefaciens BA-HZ54 strain with an accession number of CCTCC No. M 20211295, which is preserved on Oct. 20, 2021 at the China Center for Type Culture Collection (abbreviated as CCTCC) of Wuhan University, Wuhan, China, and named as Bacillus amyloliquefaciens BA-HZ54 by classification.

The Brevibacillus laterosporu is a Brevibacillus laterosporu BL-TS08 strain with an accession number of CCTCC NO: M 20211296, which is preserved on Oct. 20, 2021 at the China Center for Type Culture Collection (abbreviated as CCTCC) of Wuhan University, Wuhan, China, and named as Brevibacillus laterosporu BL-TS08 by classification.

The Bacillus mucilaginosus Krassilnikov is a Bacillus mucilaginosus Krassilnikov BM-TS05 strain with an accession number of CCTCC NO: M 20211297, which is preserved on Oct. 20, 2021 at the China Center for Type Culture Collection (abbreviated as CCTCC) of Wuhan University, Wuhan, China, and named as Bacillus mucilaginosus Krassilnikov BM-TS05 by classification.

Further, the Enterobacter ludwigii is an Enterobacter ludwigii BG10-1 strain with an accession number of CCTCC NO: M 2016014, which is preserved on Jan. 7, 2016 at the China Center for Type Culture Collection of Wuhan University (CN201610155898.9).

Further, the Myroides odoratimimus is a Myroides odoratimimus 3J2MO with an accession number of CCTCC NO: M 2017329 (CN201811409668.6), which is preserved on Jun. 13, 2017 at the China Center for Type Culture Collection of Wuhan University.

Preferably, the microbial agent has a living bacteria count of the Bacillus amyloliquefaciens being 2×10⁹ cfu/g or more, a living bacteria count of the Brevibacillus laterosporu being 2×10⁹ cfu/g or more, a living bacteria count of the Bacillus mucilaginosus Krassilnikov being 1×10¹⁰ cfu/g or more, a living bacteria count of the Enterobacter ludwigii being 1×10¹⁰ cfu/g or more, and a living bacteria count of the Myroides odoratimimus being 2×10⁹ cfu/g or more.

The above microbial strains are isolated from peanut pods and rhizospheres in main peanut production regions of China, and can effectively antagonize the growth of Aspergillus flavus and inhibit the production of aflatoxin through indoor antagonism, an in-vivo antibacterial and toxin reducing test and a field control test.

Further, the microbial agent according to the present disclosure is a highly concentrated live bacterial granule or powder or aqueous agent, preferably the granule.

According to the above solution, a particle carrier of the microbial agent of the present disclosure includes humic acid, tapioca flour and a bentonite binder. The raw materials are present in the particle carrier in a ratio of about 8.5:10:0.5. The raw materials are uniformly mixed and granulated and dried for standby application.

According to the above solution, the microbial agent of the present disclosure is produced by compounding concentrated bacterial solutions of strains with the particle carrier. Specifically, concentrated thalli obtained by centrifugation of fermentation broths of the strains are dissolved in an appropriate amount of water to be uniformly sprayed and adsorbed onto the carrier.

Provided is use of the microbial agent in field preventing and controlling aflatoxin and aflatoxigenic strain and promoting yield increase of crops.

According to the above solution, a specific application method is as follows: the microbial agent is spread onto soil or to rhizospheres of crops before sowing or during a growth period. The microbial agent is applied alone or mixed with a base fertilizer to be subjected to artificial broadcast application/hole application, or mechanically spread onto soil when applied during a sowing stage; and the microbial agent is applied alone or mixed with soil to be spread onto roots of crops when applied during the growth period.

According to the above solution, the crop includes peanuts and the like.

According to the above solution, the application time is preferably at a sowing stage or a flowering and pegging stage, and a usage amount is 2-4 kg/mu.

The beneficial effects of the present disclosure are as follows:

1. The microbial agent of the present disclosure can improve the structures of microbial populations in soil, optimize the microecological environment, remarkably inhibit the growth and reproduction of toxin-producing strain such as Aspergillus flavus in soil, reduce the risk of aflatoxin pollution in peanuts, and improve the product quality and safety level, and has significant social benefits.

2. The microbial agent of the present disclosure can reduce crop diseases, increase the number of pods per plant, and increase kernel fullness and yield, and has significant economic benefits.

3. The microbial agent of the present disclosure can promote the increase of the number of root nodules in crops, and promote crop growth and improve crop yield and quality.

4. The microbial agent of the present disclosure can be applied alone or applied with a fertilizer during the sowing stage, is convenient to store and use, is easy to promote and has a small usage amount per mu (only 2-4 kg/mu).

5. The microbial agent of the present disclosure not only avoids environmental pollution and energy waste caused by excessive chemical fertilizer input, but also facilitates improving soil fertility, and improving the farmland ecological environment, has significant ecological benefits, and is of great significance for reducing fertilizer application, peak carbon dioxide emissions and carbon neutrality. The microbial agent has a small usage amount, and is easy to use, convenient to store and easy to promote.

### BRIEF DESCRIPTION OF THE DRAWINGS

FIG. 1: Microbial agent with preventing and controlling aflatoxin and aflatoxigenic strain thereof and promoting yield increase of crops.

FIG. 2: Comparison of disease resistance of control peanuts and peanuts treated with a microbial agent.

FIG. 3: Effect of microbial agent treatment on promoting peanut yield increase.

### DESCRIPTION OF THE EMBODIMENTS

### Example 1: Preparation of a Microbial Agent with the Effects of Prevention and Control of Aflatoxin and Toxin-Producing Strain Thereof and Promotion of Crop Yield Increase

### 1. Identification of Strains

Microbial strains involved in the present disclosure were obtained from peanut pods and rhizospheres in Tangshan, Hebei and Hezhou, Guangxi by conventional bacterial isolation and purification, and molecular identification of a 16S rDNA sequence. Wherein Bacillus amyloliquefaciens BA-HZ54 with an accession number of CCTCC NO: M 20211295, Brevibacillus laterosporu BL-TS08 with an accession number of CCTCC NO: M 20211296 and Bacillus mucilaginosus Krassilnikov BM-TS05 with an accession number of CCTCC NO: M 20211297 were preserved on Oct. 20, 2021 at the China Center for Type Culture Collection (abbreviated as CCTCC) of Wuhan University. Enterobacter ludwigii BG10-1 was preserved on Jan. 7, 2016 at the China Center for Type Culture Collection of Wuhan University (CN105586300B), with an accession number of CCTCC NO: M 2016014. Myroides odoratimimus 3J2MO was preserved on Jun. 13, 2017 at the China Center for Type Culture Collection of Wuhan University, with an accession number of CCTCC NO: M 2017329 (CN201811409668.6).

### 2. Preparation of the Microbial Agent

1) Microbial fermentation production. The activated Bacillus amyloliquefaciens, Brevibacillus laterosporu, Bacillus mucilaginosus Krassilnikov, Enterobacter ludwigii and Myroides odoratimimus were separately inoculated into a liquid medium (containing 3.5-4.0% corn flour, 1.5-2.0% peptone, 0.4-0.5% K₂HPO₄+KH₂PO₄ (1:1), and 1 L of water, having a pH of 7.0-7.2, and being sterilized at 121° C. for 20 min) under aseptic conditions, and shaking culture was performed in a triangular flask at 180 r/min at 37° C. for 24 h. The cultured strains were then separately inoculated into a 300 L fermentation tank at an inoculation amount of 1% for fermentation production. The fermentative cultivation was carried out at a temperature of 30-37° C., a pH of 7.0-7.2, and a stirring speed of 180-220 rpm, and the fermentation was stopped after the amount of thalli reached 1×10¹⁰ cfu/ml.

2) Preparation of a particle carrier of the microbial agent. The particle carrier consists of humic acid, tapioca flour, and a bentonite binder. The raw materials were present in the particle carrier in a ratio of 8.5:10:0.5. The raw materials were uniformly mixed and granulated by a granulator and dried for standby application.

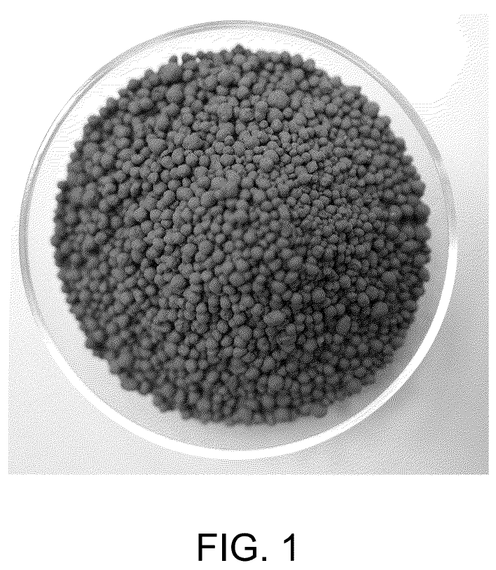

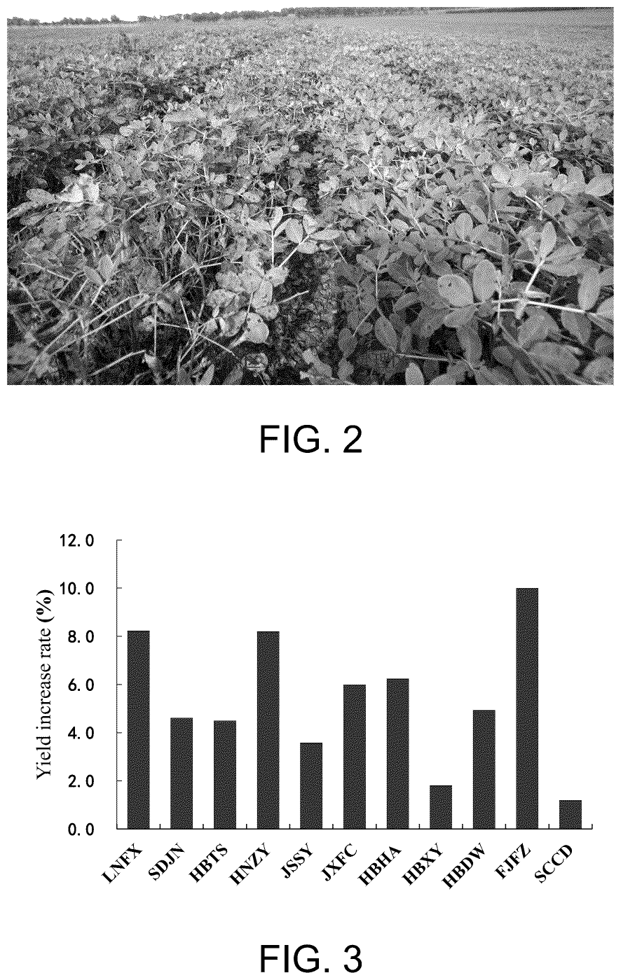

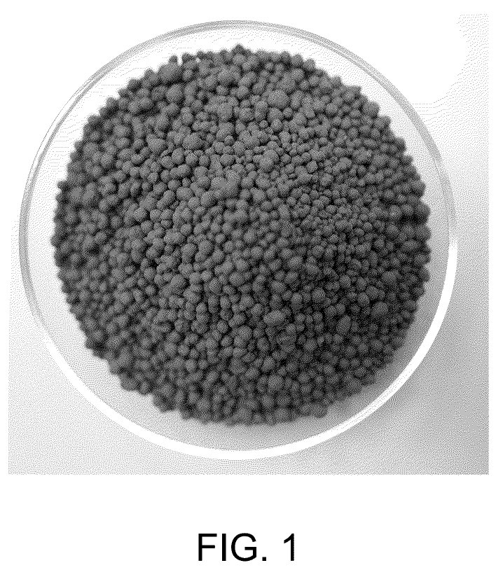

3) Preparation of a microbial agent product. According to a ratio of fermentation broths of 200 mL of Bacillus amyloliquefaciens+200 mL of Brevibacillus laterosporu+1 L of Bacillus mucilaginosus Krassilnikov+1 L of Enterobacter ludwigii+1 L of Myroides odoratimimus for producing 1 kg of a microbial fertilizer, thalli obtained by centrifugation of five strain fermentation broths with determined volumes were dissolved in an appropriate amount of water to prepare a mixed bacterial suspension, then the carrier was sprayed in a mixer (a mass ratio of the bacterial suspension to the carrier was 1:10), and drying was performed at a low temperature (≤60° C.) to prepare the microbial agent (see FIG. 1), and the microbial agent was packaged to obtain a finished microbial agent product. The microbial agent developed has a living bacteria count of the Bacillus amyloliquefaciens being 2×10⁹ cfu/g or more, a living bacteria count of the Brevibacillus laterosporu being 2×10⁹ cfu/g or more, a living bacteria count of the Bacillus mucilaginosus Krassilnikov being 1×10¹⁰ cfu/g or more, a living bacteria count of the Enterobacter ludwigii being 1×10¹⁰ cfu/g or more, and a living bacteria count of the Myroides odoratimimus being 2×10⁹ cfu/g or more.

### Example 2: Application of the Microbial Agent to Reduce the Abundance of Toxin-Producing Strain Such as Aspergillus flavus in Fields

### 1) Peanut Field Trial Setup

Field demonstration application of the microbial agent product was carried out in main peanut production regions in Henan, Shandong, Hubei, and the like of China, with a test area of 50 mu at each test point, a control group and a treatment group were set, and an isolation zone was set between the treatment group and the control group.

Control group: an area was 25 mu, a variety was a local main variety, and a local conventional sowing method, and conventional field management measures such as weeding, pest control, and vigorous growing control were used.

Treatment group: an area, a variety, a sowing method, and a field management technique were all the same as those in the control group, and on this basis, the microbial agent product was uniformly mixed with a base fertilizer at a usage amount of 2 kg/mu to be evenly spread onto soil with the fertilizer either mechanically or manually during a peanut sowing stage.

### 2) Field Soil Sample Collection

During a peanut harvest period, three peanut root rhizosphere soil samples and three peanut pod rhizosphere soil samples were randomly collected at 5-10 cm from a control area and a treatment area, and 5 sub-samples were taken per sample by five-point sampling. The 5 sub-samples were mixed into one sample, 1 kg/sample, and the abundance of toxin-producing strain such as Aspergillus flavus in the soil was to be tested after reduction of the samples by quartering.

3) Detection of the Abundance of Toxin-Producing Strain Such as Aspergillus flavus in Soil

Aspergillus flavus was isolated from the peanut soils in the control group and the treatment group, purified and identified by using the morphological and molecular biological identification methods of Aspergillus flavus (for details, see the method published in Chapter 2 of “Research on the Distribution, Virulence and Infection of Aspergillus flavus in Typical Peanut Production Areas in China”—a master's thesis of the Chinese Academy of Agricultural Sciences of which the author is Zhang Xing). And an Aspergillus flavus toxin-producing strain was detected. The detection of the abundance of toxin-producing strain such as Aspergillus flavus in the soil is shown in Table 1. A total of 59 Aspergillus flavus strains were isolated and identified from the samples in the control area, and a distribution range of Aspergillus flavus colonies was 134-1742 CFU/g, with an average value of 790 CFU/g, and a total of 18 Aspergillus flavus strains were isolated and identified from the soil samples in the treatment area, and a distribution range of Aspergillus flavus colonies was 67-603 CFU/g, with an average value of 243 CFU/g. An inhibition rate of the microbial agent on the abundance of Aspergillus flavus in soil was 43.28%-83.33%, with an average inhibition rate of 62.88%. The microbial agent of the present disclosure is shown to able to significantly inhibit the abundance of toxin-producing strain such as Aspergillus flavus in the field.

TABLE 1
Effect of microbial agent treatment on the
abundance of toxin-producing strain in soil

| | Number of strains | | Colony number | | |
|---|---|---|---|---|---|
| | (plant) | | (CFU/g) | | Inhibition |
| Test point | Control | Treatment | Control | Treatment | rate (%) |
| HNZY | 23 | 9 | 1541 | 603 | 60.87 |
| SDJN | 2 | 1 | 134 | 67 | 50.00 |
| JSSY | 6 | 1 | 402 | 67 | 83.33 |
| HBXY | 26 | 6 | 1742 | 402 | 76.92 |
| YNWS | 2 | 1 | 134 | 76 | 43.28 |
| Average | 11.8 | 3.6 | 790 | 243 | 62.88 |

### Example 3: Effect of Application of the Microbial Agent on Prevention and Control of Aflatoxin Pollution

The field trial setup was the same as that in Example 2.

### 1) Peanut Sample Collection and Treatment

Peanut pod samples were randomly collected from a control area and a treatment area during peanut harvest and dried in the sun. The samples were reduced by quartering, and then ground. 1.0 g of the samples were weighed for determination of the aflatoxin content. In addition, control and treatment samples (at least 3 replicates) at representative test points were randomly selected, and the aflatoxin content was determined after toxin-producing cultivation.

### 2) Peanut Toxin-Producing Cultivation and Aflatoxin Content Determination

Peanut powder samples were weighed into petri dishes, with 3 biological replicates per treatment, and the samples were placed in a thermostatic incubator and incubated continuously in the dark for 3.5 days at 28±1° C. and a relative humidity of 90%. After drying in a constant temperature drying oven (110° C., 1 h), 1.0 g of the peanut powder sample was weighed into a centrifuge tube after cooling, 5 ml of a 70% methanol solution (containing 4% NaCl) was added, vortexing and uniform mixing were performed, the obtained mixture was shaken on a shaker for 2 h, centrifuged at 4500 r/min, and allowed to pass through an immunoaffinity column and an organic filter membrane, and the content of aflatoxins B₁, B₂, G₁, and G₂ was detected by high performance liquid chromatography (HPLC). The total amount of aflatoxins (AFTs) is the sum of the above 4 aflatoxins.

HPLC conditions: a C18 chromatographic column (4.6 mm×150 mm, 5 μm) having a column temperature of 35° C. was used; a mobile phase of methanol:water (V:V=45:55) was used, with a flow rate of 0.8 mL/min; a post-column photochemical derivatization method using a photochemical derivatizer of 254 nm was used; a fluorescence detector (an excitation wavelength of 360 nm, and an emission wavelength of 440 nm) was used, an injection volume was 10 μl, and the determination time was 22 min.

Aflatoxin (95.7 μg/kg) was detected in one sample of the control group, while no aflatoxin was detected in the peanuts in the treatment group during harvest. The comparison of the aflatoxin content of samples from two test points under toxin-producing cultivation conditions is shown in Table 2. As can be seen from Table 2, the average aflatoxin content in the control area was 6.93 μg/kg and 11.76 μg/kg, respectively, and the average aflatoxin content of the peanuts in the treatment area was 1.24 μg/kg and 3.47 μg/kg, respectively. The effect of microbial agent treatment on controlling the content of aflatoxin in peanuts was 70% or more.

TABLE 2
Effect of microbial agent treatment on controlling aflatoxin
in peanuts under toxin-producing cultivation conditions

| | | | AFB1 | AFT | |
|---|---|---|---|---|---|
| | | AFT | exceeding | content | AFT |
| | | detection | standard | average | inhibition |
| Test point | | rate (%) | rate (%) | (μg/kg) | rate (%) |
| HNZY | Control | 100 | 0 | 11.76 | 70.5 |
| | Treatment | 80 | 0 | 3.47 | |
| JXFC | Control | 100 | 33.3 | 6.93 | 82.1 |
| | Treatment | 88.9 | 0 | 1.24 | |

### Example 4: Application of the Microbial Agent to Increase Peanut Yield

The field trial setup was the same as that in Example 2.

Biological and economic traits such as leaf color, resistance, full pods, 100-pod weight, and yield per mu in the control and treatment areas were respectively examined at test and demonstration points such as Henan, Shandong, Hebei, and Hubei.

And nodulation was investigated and determined. Compared with the control group, a root system of peanuts treated with the microbial agent was more developed, and grew more vigorously, and the decline was delayed; the number of root nodules increased significantly, and increased by 30 times or more, and the cumulative nitrogenase activity increased by more than 50 times, indicating that the microbial agent of the present disclosure can effectively promote the nodulation and nitrogen fixation of peanut roots.

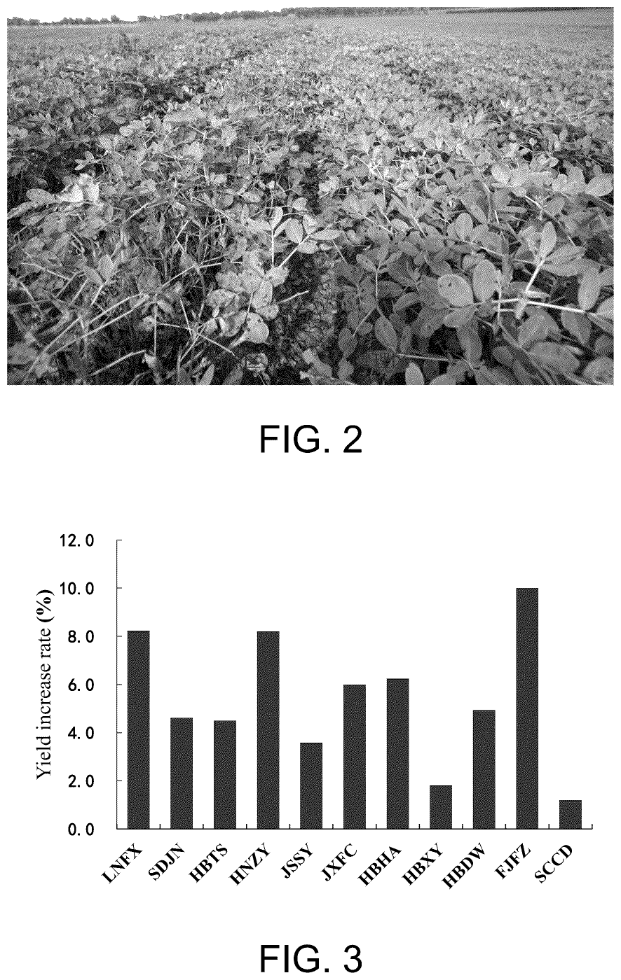

It was found that compared with the control area, the growth vigour of the peanuts was stronger, the leaves were more dense and greener, and the resistance to leaf spot disease, and the like was enhanced in the microbial agent treatment area (FIG. 2). Yield determination results in 2020 (FIG. 3): the yield of peanuts per mu at each test point increased by 5-25 kg, with a yield increase rate of 1.2-10.0%, and the average yield per mu increased by 15.2 kg, with an average yield increase rate of 5.59%; and the yield of peanuts per mu at each test point increased by 6.67-43 kg in 2021, with a yield increase rate of 2.2%-25.0%, and the average yield per mu increased by 37.88 kg, with an average yield increase rate of 13.69%.

The determination results of economic traits in representative test points are shown in Table 3: compared with the control group, the number of pods per plant and the 100-pod weight of the peanuts treated with the microbial agent increased by 27.6% and 10.94%, respectively, and full peanut pods were promoted, and a full pod rate increased by 7.45%, thereby promoting the increase of the peanut yield.

The results show that microbial agent treatment has the effects of enhancing peanut disease resistance, increasing the number of peanut pods per plant, promoting kernel fullness, and increasing peanut pod weight per plant, 100-pod weight and peanut yield.

TABLE 3
Effect of microbial agent treatment
on promoting peanut yield increase

| | | Number of | Full | | | Yield |
|---|---|---|---|---|---|---|
| | | pods per | pod | 100-pod | Yield | increase |
| Test | | plant | rate | weight | per mu | rate |
| point | Sample type | (pcs) | (%) | (g) | (kg) | (%) |
| HBXY | Control | 20.00 | 85.07 | 178.00 | 291.3 | 19.4 |
| | Demonstration | 31.33 | 85.84 | 194.00 | 347.9 | |
| LNFX | Control | 36.00 | 61.00 | 170.00 | 400.2 | 25 |
| | Demonstration | 38.00 | 73.00 | 172.03 | 500.2 | |
| JLFY | Control | 15.70 | 89.73 | 151.80 | 310.1 | 6.12 |
| | Demonstration | 17.05 | 91.25 | 155.65 | 329.1 | |
| GDZJ | Control | 14.60 | 74.00 | 143.30 | 230.6 | 18.5 |
| | Demonstration | 16.80 | 83.30 | 158.10 | 273.3 | |
| HBDW | Control | 9.40 | 79.80 | 149.36 | 182.5 | 7.84 |
| | Demonstration | 9.50 | 82.10 | 160.26 | 196.8 | |
| HNZY | Control | 21.90 | 82.60 | 201.25 | 236.0 | 18.27 |
| | Demonstration | 32.70 | 87.50 | 211.58 | 279.2 | |
| FJFZ | Control | 9.83 | 83.29 | 205.10 | 296.6 | 2.22 |
| | Demonstration | 11.92 | 87.92 | 206.87 | 303.2 | |
| YNWS | Control | 19.60 | 69.30 | 89.7 | 264.7 | 7.8 |
| | Demonstration | 32.00 | 76.50 | 135.6 | 285.3 | |

The microbial agent developed by mixing Bacillus amyloliquefaciens, Brevibacillus laterosporu, Bacillus mucilaginosus Krassilnikov, Enterobacter ludwigii and Myroides odoratimimus according to the present disclosure has the effects of preventing and controlling aflatoxin and aflatoxigenic strain thereof of peanuts and promotion of peanut yield increase. After the field application of the microbial agent, the abundance of toxin-producing strain such as Aspergillus flavus in the field can be effectively reduced, the risk of aflatoxin pollution in peanuts can be reduced, the quality and safety level of peanuts can be improved, and peanut podding can be promoted, kernel fullness and yield can be improved, and significant social, economic, and ecological benefits can be produced, which is of great significance for improving the quality and efficiency of the peanut industry, and promoting green and high-quality development, and has broad application prospects.

What is claimed is:

1. A microbial agent with functions of preventing and controlling aflatoxin and aflatoxigenic strain thereof and promoting yield increase of crops, compounded from 5 microbes comprising Bacillus amyloliquefaciens, Brevibacillus laterosporu, Bacillus mucilaginosus Krassilnikov, Enterobacter ludwigii and Myroides odoratimimus by separate fermentative cultivation, concentration and mixing; the Bacillus amyloliquefaciens is a Bacillus amyloliquefaciens BA-HZ54 strain with an accession number of CCTCC NO: M 20211295; the Brevibacillus laterosporu is a Brevibacillus laterosporu BL-TS08 strain with an accession number of CCTCC NO: M 20211296; and the Bacillus mucilaginosus Krassilnikov is a Bacillus mucilaginosus Krassilnikov BM-TS05 strain with an accession number of CCTCC NO: M 20211297.

2. The microbial agent according to claim 1, wherein the Enterobacter ludwigii is an Enterobacter ludwigii BG10-1 strain with an accession number of CCTCC NO: M 2016014.

3. The microbial agent according to claim 1, wherein the Myroides odoratimimus is a Myroides odoratimimus 3J2MO strain with an accession number of CCTCC NO: M 2017329.

4. The microbial agent according to claim 1, wherein the microbial agent has a living bacteria count of the Bacillus amyloliquefaciens being 2×10⁹ cfu/g or more, a living bacteria count of the Brevibacillus laterosporu being 2×10⁹ cfu/g or more, a living bacteria count of the Bacillus mucilaginosus Krassilnikov being 1×10¹⁰ cfu/g or more, a living bacteria count of the Enterobacter ludwigii being 1×10¹⁰ cfu/g or more, and a living bacteria count of the Myroides odoratimimus being 2×10⁹ cfu/g or more.

5. The microbial agent according to claim 1, wherein the microbial agent is a highly concentrated live bacterial granule or powder or aqueous agent.

6. The microbial agent according to claim 5, wherein a particle carrier of the microbial agent comprises humic acid, tapioca flour and a bentonite binder; and the microbial agent is produced by compounding concentrated bacterial solutions of strains with the particle carrier.

7. Use of the microbial agent according to claim 1 in field preventing and controlling aflatoxin and aflatoxigenic strain and promoting yield increase of crops.

8. The use according to claim 7, wherein the microbial agent is spread onto soil or to rhizospheres of crops before sowing or during a growth period.

9. The use according to claim 7, wherein the application time is preferably at a sowing stage or a flowering and pegging stage, and a usage amount is 2-4 kg/mu.

10. The use according to claim 7, wherein the crops comprise peanuts.
